(19) United States
(12) Patent Application Publication
(10) Pub. No.: US 2026/0176561 A1
(43) Pub. Date: Jun. 25, 2026

(54) Bioreactor Systems and Methods of Operating Same

(71) Applicant: National Resilience, Inc., San Diego, CA (US)

(72) Inventors: Huanchun Cui, San Diego, CA (US); Thomas Erdenberger, San Diego, CA (US); Thomas Ransohoff, San Diego, CA (US)

(21) Appl. No.: 19/124,766

(22) PCT Filed: Oct. 25, 2023
(86) PCT No.: PCT/US2023/077732

§ 371 Date: Apr. 25, 2025

Related U.S. Application Data
(60) Provisional application No. 63/381,096, filed on Oct. 26, 2022.

(51) Int. Cl. C12M 3/06; B04B 5/04

(52) U.S. Cl. CPC C12M 23/16; B04B 5/0414; B04B 5/0421; B04B 5/0442

(57) ABSTRACT

The systems and methods disclosed herein can generate product from cells using a bioreactor system. The bioreactor system can include a centrifuge. The bioreactor system can use a centrifuge to improve product yield. The centrifuge can be used to separate content received from the bioreactor. The separated content can be diverted back to the bioreactor, or filtered and/or collected.

### CROSS-REFERENCE TO RELATED APPLICATIONS

This application claims priority to U.S. Provisional Patent Application No. 63/381,096, filed on Oct. 26, 2022, which is incorporated herein by reference in its entirety.

### BACKGROUND

Cells can be used to generate therapeutic products. Bioreactors can grow large quantities of cells and can induce cells to generate therapeutic products in large batches. Producing large amounts of products is often dependent on the ability to run a bioreactor for extended periods of time, as well as the overall viability of cells. Bioreactors may suffer from product loss if their configuration or setup leads to inefficient operations.

### SUMMARY

In an aspect, the present disclosure provides a system comprising: a centrifuge in fluidic communication with a bioreactor, wherein the centrifuge is configured to receive and separate content from the bioreactor into a first portion and a second portion, wherein a composition of the first portion is different from a composition of the second portion; a first fluidic pathway configured to transfer the first portion from the centrifuge back to the bioreactor; and a second fluidic pathway configured to transfer the second portion from the centrifuge to a filtration unit.

In some embodiments, the centrifuge comprises an inlet port configured to receive content from the bioreactor. In some embodiments, the centrifuge comprises a first outlet port connected to the first fluidic pathway, and a second outlet port connected to the second fluidic pathway. In some embodiments, the content in the bioreactor has a cell concentration ranging from 20 million to 150 million cells per milliliter. In some embodiments, a cell concentration of the first portion is higher than the cell concentration of the content in the bioreactor. In some embodiments, a cell concentration of the first portion is based at least on the cell concentration of the content in the bioreactor and a spin force that the centrifuge exerts on content received by the centrifuge. In some embodiments, the second portion comprises a supernatant. In some embodiments, the centrifuge comprises plastic. In some embodiments, the centrifuge is configured for single use. In some embodiments, the centrifuge comprises an aseptic environment. In some embodiments, the centrifuge is configured to be operated continuously for an extended time duration of at least 10 days. In some embodiments, the centrifuge is disposable and/or replaceable. In some embodiments, the centrifuge is releasably coupled to the bioreactor. In some embodiments, the centrifuge is releasably coupled to the filtration unit. In some embodiments, the content in the bioreactor comprises a plurality of cells, and wherein the first portion comprises at least 50% of a number of cells that are packed when content is received by the centrifuge. In some embodiments, the first fluidic pathway comprises a bleeding port. In some embodiments, the bleeding port is configured to remove a predefined volume of the first portion from the first fluidic pathway before a remaining volume of the first portion is transferred back to the bioreactor. In some embodiments, the predefined volume of the first portion is determined based on a volume of the content in the bioreactor. In some embodiments, the predefined volume of the first portion is removed at a rate ranging from 1% to 30% of the volume of the content in the bioreactor per day. In some embodiments, the volume of the content in the bioreactor is less than or equal to a vessel volume size of the bioreactor. In some embodiments, the system further comprises the bioreactor. In some embodiments, the bioreactor is configured to receive media at a predefined rate per day. In some embodiments, the predefined rate is based on a volume of the content in the bioreactor, a predefined volume of the first portion that is removed through bleeding, and a volume of perfusate generated by the filtration unit. In some embodiments, the predefined rate ranges from 50% to 1000% of the volume of the content in the bioreactor per day. In some embodiments, the content in the bioreactor has a cell concentration ranging from 20 million to 150 million cells per milliliter. In some embodiments, the system further comprises the filtration unit. In some embodiments, the filtration unit is configured to enable tangential flow filtration (TFF) of the second portion to generate a perfusate and a retentate. In some embodiments, the perfusate is removed at a predefined volumetric rate per day. In some embodiments, the predefined volumetric rate is based on a volume of the content in the bioreactor. In some embodiments, the predefined volumetric rate ranges from 50% to 1000% of the volume of the content in the bioreactor. In some embodiments, the system further comprises a third fluidic pathway configured to transfer the retentate from the filtration unit back to the bioreactor for re-use in production of a product. In some embodiments, the system is used for protein production.

In some embodiments, the system is configured to be operated continuously for an extended time duration. In some embodiments, the extended time duration is at least 10 days. In some embodiments, the content in the bioreactor is maintained having a viable cell density of at least 20 million cells per milliliter.

In an aspect, the present disclosure provides a method comprising: performing centrifugation on content received from a bioreactor, thereby separating the received content into a first portion and a second portion, wherein a composition of the first portion is different from a composition of the second portion; and transferring the first portion back to the bioreactor, and transferring the second portion to a filtration unit.

In some embodiments, the content comprises a cell concentration ranging from 20 million to 150 million cells per milliliter. In some embodiments, a cell concentration of the first portion is higher than the cell concentration of the content in the bioreactor. In some embodiments, a cell concentration of the first portion is based at least on the cell concentration of the content in the bioreactor and a spin force exerted by the centrifugation on the received content. In some embodiments, the second portion comprises supernatant. In some embodiments, the received content is separated such that the first portion comprises at least 50% of a number of cells that are packed by the centrifugation. In some embodiments, the method further comprises: removing a predefined volume of the first portion before transferring a remaining volume of the first portion back to the bioreactor. In some embodiments, the predefined volume of the first portion is determined based on a volume of the content in the bioreactor. In some embodiments, the predefined volume of the first portion is removed at a rate ranging from 1% to 30% of the volume of the content in the bioreactor per day. In some embodiments, the bioreactor receives media at a predefined rate per day. In some embodiments, the predefined rate is based on a volume of the content in the bioreactor, a predefined volume of the first portion that is removed through bleeding, and a volume of perfusate generated by the filtration unit. In some embodiments, the predefined rate ranges from 50% to 1000% of the volume of the content in the bioreactor. In some embodiments, the content comprises a cell concentration ranging from 20 million to 150 million cells per milliliter. In some embodiments, the method further comprises: performing tangential flow filtration (TFF) on the second portion using the filtration unit to thereby generate a perfusate and a retentate. In some embodiments, the method further comprising: removing the perfusate at a predefined volumetric rate. In some embodiments, the predefined volumetric rate is based on a volume of the content in the bioreactor. In some embodiments, the predefined volumetric rate ranges from 50% to 1000% of the volume of the content in the bioreactor. In some embodiments, the method further comprises: transferring the retentate from the filtration unit back to the bioreactor for re-use in production of a product. In some embodiments, the method is used for protein production.

In another aspect, the present disclose provide a non-transitory computer readable medium comprising instructions that, when executed by a computer processor, cause the computer processor to control a system described in this disclosure.

In another aspect, the present disclosure provides a non-transitory computer readable medium comprising instructions that, when executed by a computer processor, cause the computer processor to perform methods described in this disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1 shows a schematic of an exemplary bioreactor system without a centrifuge.

FIG. 2 shows a schematic of an exemplary bioreactor system including a centrifuge.

FIG. 3 shows a computer control system that is programmed or otherwise configured to implement methods provided herein.

### DETAILED DESCRIPTION

While various embodiments have been shown and described herein, it will be obvious to those skilled in the art that such embodiments are provided by way of example only. Numerous variations, changes, and substitutions can occur to those skilled in the art without departing from the present disclosure. It should be understood that various alternatives to the embodiments described herein can be employed.

Provided herein are systems and methods for generating cells and products derived from cells. The products generated using the systems and methods herein can be used to treat or ameliorate a disorder or disease in a subject. The systems and methods can improve efficiency in the generation of cells and products derived from cells.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

Bioreactors can be used to generate large quantities of cells. The cells can be used to produce products or macromolecules that have a therapeutic benefit, or can be used as reagents for experimental assays, for example. The bioreactors can include a vessel for incubating cells. Bioreactors can be operated in a continuous manner, or fed-batch manner, for example. The bioreactor can include an inlet that allows for fresh media to be added to the vessel (of the bioreactor). The bioreactor can include an outlet that allows for media (e.g., spent media) to exit the bioreactor. For continuous mode of operation the input of fresh media and removal of media allows for the continuous flow of fresh media into the vessel of the bioreactor while maintaining the same volume and/or conditions for cell growth. For fed-batch operation medium is added to the bioreactor but is not removed during culture growth. For continuous mode of operation as cells grow and the overall amount (or density) of the cells in the bioreactor increases, medium containing the cells may exit through the outlet and fresh media can be added to the vessel. In this way, the cell density can be maintained in a range that is suitable for efficient cell growth. Without the input of additional media and nutrients, and maintenance of a threshold cell density, the viability of the cells in the bioreactor may be affected negatively, resulting in a reduction of cell viability. The reduction of cell viability, in turn, can lead to a reduction in the yield of cells and/or product produced by cells for further processing.

Bioreactor productivity can be improved by maintaining high viable cell density in the bioreactor. To maintain high viable cell density for an extended period of time in the bioreactor, perfusion can be adopted where spent media is being removed from the bioreactor and fresh media is being fed into the bioreactor. In order to increase viable cell density (e.g., 20 million to 150 million/mL and prolong perfusion duration (e.g., greater than 10 days), bioreactor bleeding can be implemented where bioreactor content is removed. However, such bleeding strategy can lead to product loss as the bled media also comprises viable cells and/or product. Further, at increasingly high cell densities, performance of microfiltration methods of separating the cells from the medium during perfusion may decrease and the microfilter may be more prone to fouling.

The present disclosure provides systems and methods for increasing the efficiency of the production of viable cells and/or product. For example, a centrifuge coupled with a bioreactor can perform a centrifugation step. The centrifuge can be used to separate bioreactor content into two parts: (i) concentrated cells and (ii) supernatant. In some implementations, the supernatant can be further filtered for further processing. In some implementations, the concentrated cells can be returned to the bioreactor for reuse.

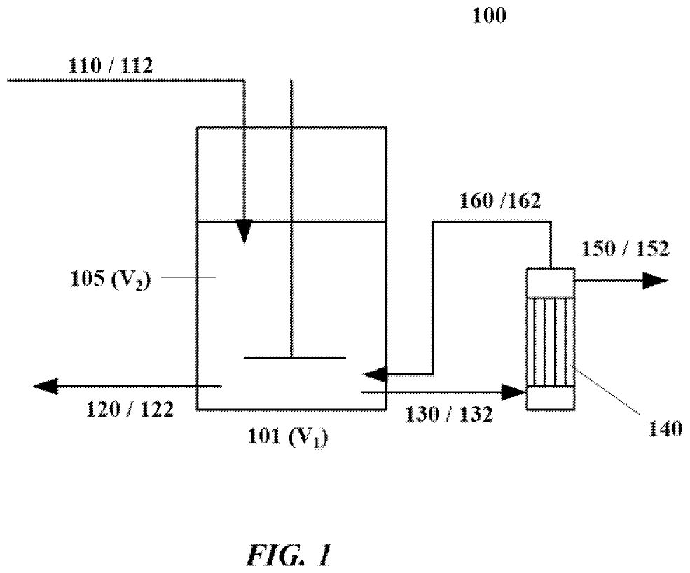

FIG. 1 shows an exemplary bioreactor system 100 without a centrifuge. Bioreactor 101 includes a vessel with a volume V₁. The vessel can contain media 105 at a volume V₂ for growing cells, for example. Bioreactor 101 can include various components and/or devices that are configured to regulate temperature, regulate pressure, aerate the media, and/or agitate the cells, to provide an environment for cell growth or other processes. Cells in the bioreactor 101 can generate and secrete a product into the media that is collected via other processes in the system. Input line 110 can be used to add fresh media 112 to the bioreactor 101, for example, to supply cells in the bioreactor with nutrients.

The bioreactor 101 can include one or more outlet lines, such as outlet line 130 that can direct cell culture 132 to a filtration unit 140. The filtration unit 140 can be used to separate the cell culture 132 into a perfusate portion 152 and a retentate portion 162. The retentate portion 162 includes cells, while the perfusate 152 includes product (preferably being substantially free of cells) for further processing. The perfusate 152 can be collected via fluidic pathway 150 and the product can be isolated from the perfusate 152. The retentate 162 including cells can be returned to the bioreactor 101 via fluidic pathway 160, for example, to allow for the cells to continue to grow and generate product.

The bioreactor 101 can also include an outlet line to a collection unit. The collection unit can be used to collect cell culture from the bioreactor and the cell culture can be used for further processing. For example, the cells can be collected in the collection unit and removed from the system. In another example, the cells can be collected in a collection unit, have processes performed on the cell culture, and then the cell culture can be directed to filtration unit 140 from the collection unit. The processes, for example, can include monitoring the cells in the collection unit. The cells can be monitored for cell viability and the collected cell culture that demonstrates cell viability below a threshold can be removed. Cell culture collected in a collection unit that demonstrates cell viability above a threshold can be directed to the filtration unit 140.

The bioreactor 101 can also include a bleeding port or line 120 which can be used to remove a portion of the bioreactor content 122 to be discarded, for example. Bleeding of the bioreactor content can facilitate the removal of spent media as well as removing excess cells to maintain a suitable cell density in the bioreactor for high cell viability. Since the placement of the bleeding port 120 directly connects to the bioreactor, however, the bled bioreactor content may include viable cells and/or product. The input of media into the bioreactor and removal of content from the bioreactor can be balanced to maintain a relatively constant volume in system 100. For example, fresh media 112 can be added to the bioreactor 101 at a defined rate of 0.5-20 bioreactor volumes (V)/day (e.g., 1000 L to a 1000 L bioreactor=1.0V/day.) For example, to maintain a constant volume in the system, fresh media can be added at a defined rate, e.g., 120% V/day, a portion of the bioreactor content can exit as perfusate 152, e.g., 100% V/day, and a portion of the bioreactor content, e.g., 20% V/day, can exit the bioreactor via the bleeding port 120.

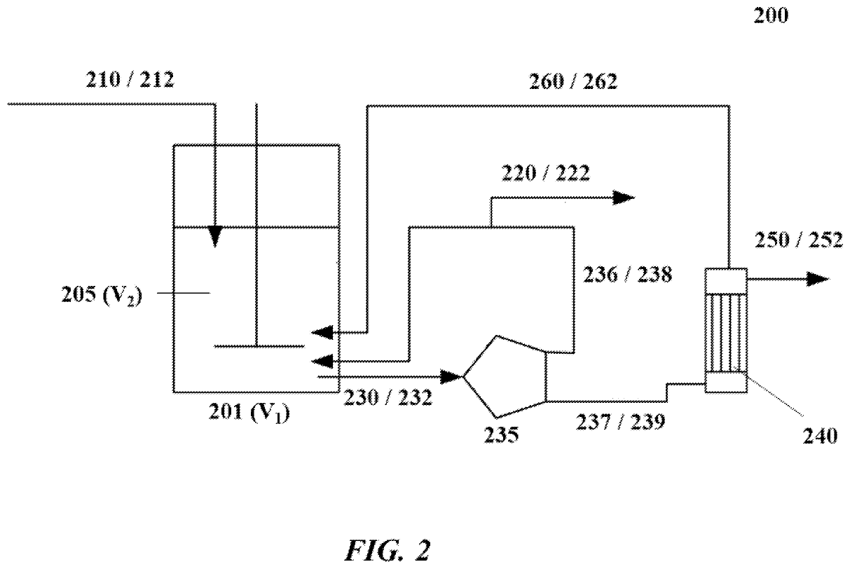

A bioreactor system that includes a centrifuge as described herein can reduce the loss of viable cells and/or product, improve filtration, and increase product yield. FIG. 2 shows an exemplary system 200 including a bioreactor and a centrifuge. Bioreactor 201 includes a vessel with a volume V₁. The vessel can contain media 205 of volume V₂ for growing cells, for example. Bioreactor 201 can include various components and/or devices that are configured to regulate temperature, regulate pressure, aerate the media, and/or agitate the cells, to provide an environment for cell growth or other processes. Cells in the bioreactor 201 can produce a product within its confines and secrete a product into the media that is collected via other processes in the system. Input line 210 can be used to add fresh media 212 to the bioreactor 201, for example, to supply cells in the bioreactor with nutrients.

The bioreactor 201 can include one or more outlet lines, such as outlet line 230 that can direct cell culture 232 to a centrifuge 235. The centrifuge 235 in in fluidic communication with the bioreactor 201. The centrifuge 235 can be configured to receive and separate content from the bioreactor 201, for example, into a first portion and a second portion, where the composition of the first portion is different from a composition of the second portion. In some implementations, the centrifuge 235 can apply a spin force to the received bioreactor content to generate a cell comprising portion 238 (e.g., a concentrate, pellet, etc.) and a supernatant portion 239 (e.g., centrate). A first fluidic pathway 236 can be configured to transfer the first portion (e.g., cell comprising portion 238) from the centrifuge back to the bioreactor 201. A second fluidic pathway (e.g., fluidic pathway 237) can configured to transfer the second portion (e.g., supernatant portion 239) from the centrifuge 235 directly to a filtration unit 240.

The bioreactor 201 can also include an outlet line to a collection unit. The collection unit can be used to collect cell culture from the bioreactor and the cell culture can be used for further processing. For example, the cells can be collected in the collection unit and removed from the system. In another example, the cells can be collected in a collection unit, have processes performed on the cell culture, and then the cell culture can be directed to the centrifuge 235 from the collection unit. The processes, for example, can include monitoring the cells in the collection unit. The cells can be monitored for cell viability and the collected cell culture that demonstrates cell viability below a threshold can be removed. Cell culture collected in a collection unit that demonstrates cell viability above a threshold can be directed to the centrifuge 235.

The filtration unit 240 can be used to separate the supernatant portion 239 into a perfusate portion 252 and a retentate portion 262. The retentate portion 262 includes cells, while the perfusate 252 includes product (preferably being substantially free of cells) for further processing. The perfusate 252 can be collected via fluidic pathway 250 and the product can be isolated from the perfusate 252. The retentate 262 including cells can be returned to the bioreactor 201 via fluidic pathway 260, for example, to allow for the cells to continue to grow and generate product.

The system 200 can also comprise a bleeding port or line 220 which can be used to remove a portion of the system content 222. For example, the bleeding can facilitate the removal of spent media as well as removing excess cells to maintain a suitable cell density in the bioreactor for high cell viability. In some implementations, the bleeding can be configured to maintain a cell concentration ranging from 20 million to 150 million cells per milliliter.

The bleeding port 220 in system 200 can be placed in the fluidic pathway 236 between centrifuge 235 and bioreactor 201 that transfers the cell comprising portion 238 back to the bioreactor, as shown in FIG. 2. Since the cell comprising portion 238 includes less product compared to the cell culture 232 transferred from the bioreactor 201 to the centrifuge 235, the removal of system content 222 from the bleeding port 220 results in less product loss. Additionally, as the centrifuge 235 diverts the cell comprising portion 238 away from the filtration unit 240, the cell count in supernatant portion 239 transferred to the filtration unit 240 is significantly reduced. A reduction of cells transferred to the filtration unit 240 may reduce or prevent fouling and clogging of the filtration unit 240, which may extend the lifetime of the filtration unit 240. A bleeding port (e.g. 220) can also be placed in the system along fluidic pathway 260 between filtration unit 240 and bioreactor 201 that transfers the retentate portion 262 back to the bioreactor 201. As the retentate portion 262 comprises some cells and media, the bleeding from the fluidic pathway 260 can facilitate the removal of spent media as well as removing excess cells to maintain a suitable cell density in the bioreactor. Since the retentate portion 262 includes less product compared to the cell culture 232, the removal of system content from the bleeding port results in less product loss. The rates at which media is added to the bioreactor and content removed from the bioreactor system can be balanced to maintain a constant working volume in the bioreactor system 200. For example, fresh media can be added at a rate of 120% V₁/day and to maintain a constant volume, 100% V/day can exit the system as perfusate 252 and 20% V₁/day can exit the system via the bleeding port 220 (e.g., as waste). The rates of adding and removing liquid to and from the system can be predefined, fixed, and/or adjustable (manually or automatically).

While FIG. 2 describes an exemplary configuration of a bioreactor system including a bioreactor and a centrifuge, the present disclosure is not intended to be limited by any such example. Numerous variations, changes, and substitutions will occur to those skilled in the art without departing from the disclosure. For example, the entire content of a fluidic pathway exiting the centrifuge (e.g., fluidic pathway 236) can be bled through a bleeding port (e.g., bleeding port 220) without returning any of this content back to the bioreactor (which may eliminate the need for a return line from the centrifuge to the bioreactor.)

In various aspects, the systems and methods herein include a bioreactor or use/operate a bioreactor (such as bioreactor 201). The bioreactor can be used for growing or proliferating cells. The bioreactor can include multiple components that allow for cells to proliferate. The bioreactor can include a vessel (rigid or flexible container) that can hold the cells and associated fluids. The bioreactor can include cell growth media or nutrients to allow the cells to proliferate. Generally, to facilitate and maintain the growth of cells, bioreactors can include components and/or devices that are configured to regulate the temperature, pressure, pH, and nutrient level of the media in the bioreactor. The bioreactor can include an agitator and/or any other apparatus that allows the fluids in the bioreactor to flow and/or mix. Cells in the bioreactor need nutrients in order to survive, and an agitator can allow for uniform mixing of cells and nutrients throughout the bioreactor. For example, an impeller or other rotating blades can be used to mix the fluid in the bioreactor. The bioreactor can include an aeration system to introduce oxygen or other gases into the system. Aerobic organisms can use oxygen to respirate, and appropriate oxygen levels can improve the viability of the cells in the bioreactor. The aeration system can include a sparger. A sparger can be used to introduce air into the vessel via pipes connected to the bioreactor vessel. The aeration system can include baffles, which can be used to improve aeration. The baffles, which can include multiple protrusions attached to the interior of a bioreactor vessel, can disrupt vortex formation in the bioreactor. The bioreactor can include a temperature regulation apparatus, such as heater, jacket or bath. A heater can be connected to the vessel to produce heat and alter the temperature of the vessel. Similarly, a jacket can be used as a heat sink to reduce temperature by providing a material having or at a lower temperature in the jacket surrounding the vessel.

The bioreactor (such as bioreactor 201) can include various sensors that monitor the condition and growth of cells. For example, the bioreactor can include a temperature sensor (e.g., thermometer), a pressure sensor, a pH sensor, an oxygen sensor, carbon dioxide sensor and/or a cell density sensor. The sensor(s) can monitor metabolites or nutrients of the media in the bioreactor. The sensor(s) can be used to identify parameters that fall outside of desired ranges. A regulator or controller can be initialized to correct or alter the parameters, through addition of reagents and/or modulation of one or more components and/or devices of the bioreactor. For example, a temperature sensor can detect that the media of the bioreactor is at a temperature lower than a set range. The controller can initiate a heating unit to increase the temperature of the media in the bioreactor. A sensor can also detect the pressure in the bioreactor. A sensor can also detect the cell density of the contents in the bioreactor. Cell viability can be correlated with cell density, with a lower viability occurring when a cell density is too high or too low. Additionally, generation of product may be reduced at a lower cell density, and there is a waiting time/period for cells to proliferate to a higher cell density before collection of a product can improve product yield. The cell density measurement can be used to determine and/or control the volume of bleeding.

The bioreactor can include an inlet port(s) (e.g. inlet port 210) that can be used to add media to the bioreactor. The bioreactor can be configured to receive media at a predefined or variable rate per day. The bioreactor can include various input and output ports for moving media into the bioreactor and content out of the bioreactor. The output ports can be connected to fluidic pathways (e.g., 230, 236, or 237) to move the content from the bioreactor to other components and/or devices of the bioreactor system and allow for additional processes to be performed on bioreactor content. The overall volume (V₃) of the bioreactor system content can be modulated based on the volume input or removed from the bioreactor system. Generally, the overall volume of content (V₂) in the bioreactor can be held constant by balancing the bioreactor system input volume with the bioreactor system output volume. Various fluidic pathways (such as fluidic pathway 236) can be used to circulate a portion of the bioreactor output back to the bioreactor. As described herein, content from the bioreactor can be subjected to centrifugation, and a portion of the content can be returned to the bioreactor. A portion of the recirculated content can be removed (or bled) from the bioreactor system, thereby resulting in a net loss of volume in the bioreactor. As such, the rate that media is added to the bioreactor can be dependent on the volume that is removed (e.g., via bleeding or collection of perfusate) from the bioreactor. For example, a predefined rate of media added to the bioreactor can be based on a volume of the content in the bioreactor, a predefined volume of the cell comprising portion that is removed (e.g., through bleeding), and a volume of perfusate generated by the filtration unit. In some embodiments, the predefined rate of addition can range from 50%-1000% of the volume of the content in the bioreactor. The predefined rate of addition can be at least 50% of the volume of the content in the bioreactor per day. The predefined rate of addition can be at least 50%, 60%, 70%, 80%, 90%, 100%, 110%, 120%, 130%, 140%, 150%, 160%, 170%, 180%, 190%, 200%, 300%, 400%, 500%, 600%, 700%, 800%, 900%, 1000%, or more, of the volume of the content in the bioreactor per day.

The bioreactor can be used to generate cells. The cells in the bioreactor can proliferate to a particular concentration of cells. For example, the bioreactor content can have a cell concentration ranging from 20 million to 150 million cells per milliliter. The bioreactor content can have a cell concentration of at least 20 million cells per milliliter, 30 million cells per milliliter, 40 million cells per milliliter, 50 million cells per milliliter, 60 million cells per milliliter, 70 million cells per milliliter, 80 million cells per milliliter, 90 million cells per milliliter, 100 million cells per milliliter, 110 million cells per milliliter, 120 million cells per milliliter, 130 million cells per milliliter, 140 million cells per milliliter, 150 million cells per milliliter, 160 million cells per milliliter, 170 million cells per milliliter, 180 million cells per milliliter, 190 million cells per milliliter, 200 million cells per milliliter, or more. The bioreactor content can have a cell concentration of no more than 20 million cells per milliliter, 30 million cells per milliliter, 40 million cells per milliliter, 50 million cells per milliliter, 60 million cells per milliliter, 70 million cells per milliliter, 80 million cells per milliliter, 90 million cells per milliliter, 100 million cells per milliliter, 110 million cells per milliliter, 120 million cells per milliliter, 130 million cells per milliliter, 140 million cells per milliliter, 150 million cells per milliliter, 160 million cells per milliliter, 170 million cells per milliliter, 180 million cells per milliliter, 190 million cells per milliliter, 200 million cells per milliliter, or less. The cell concentration of the bioreactor can be maintained over an extended period of time. For example, the cell concentration of the bioreactor can be maintained over the entire period of operation of the bioreactor.

In various aspects, a centrifuge (e.g., centrifuge 235) is used in conjunction with a bioreactor. The centrifuge can allow for separation of a suspension of cells into a supernatant fraction and a cell fraction. The centrifuge can apply a spin force that acts on the cells of the suspension. By applying the spin force to the suspension, a greater force is applied to the cells as opposed to materials and molecules of lower density. (such as those in the buffer solution or macromolecules generated by the cells). The spin force allows for the cells to be removed from the suspension to form a first fraction. The less dense materials, which are affected less by the spin force remain in the solution and form a second fraction, corresponding to the supernatant which may have substantially less cells. The supernatant fraction and cell fraction can then be subjected to different processes after separation.

The centrifuge (e.g., centrifuge 235) can include an inlet port configured to receive content from the bioreactor. The inlet port can allow for a cell comprising suspension from the bioreactor to be transferred to the centrifuge. This can allow for the suspension to be subjected to spin force and separated into a cell comprising portion (e.g., 238) and a supernatant portion (e.g., 239). The centrifuge can include one or more outlet ports to remove content from the centrifuge. For example, the centrifuge can include an outlet port that removes the supernatant from the centrifuge. The outlet port(s) can be connected to fluidic pathway(s) to move content of the centrifuge to a different part of the system. For example, the centrifuge can include a first outlet port connected to a first fluidic pathway (e.g., fluidic pathway 236), and a second outlet port connected to a second fluidic pathway (e.g., fluidic pathway 237.) For example, the first outlet port can provide an outlet for the cell comprising portion (e.g., 238) and the second outlet port can provide an outlet for the supernatant (e.g., 239). The cell comprising portion and supernatant can be taken to different locations in the bioreactor system, or subjected to different processes via transport in different fluidic pathways. Since the supernatant can include product secreted by the cells, the supernatant can be harvested or collected and subjected to downstream processes that purify or isolate the product from the supernatant. Cells that are still viable can continue to proliferate and generate product, and can be diverted back to the bioreactor. An inlet port can also be used as an outlet port by reversing the flow of the fluid. For example, a centrifuge can include an inlet port through which the cell comprising suspension can enter a vessel or chamber of the centrifuge. A spin force can be applied to the vessel and allow for the less dense objects (e.g., supernatant) to flow out of an outlet port and maintain the more dense objects (e.g., cells) within a vessel. The flow direction can be reversed and allow for the more dense portion to exit out of an inlet port.

The centrifuge can include a rotor. The rotor can be spun to generate a spin force on the contents in the centrifuge. The rotor can include cavities or holes to hold liquid contents. The cavities or holes can hold vessels, such as bottles or chambers (rigid or flexible), which in turn can hold liquid contents. The cavities, holes, or vessels can be filled with contents from the bioreactor and subsequently subjected to a spin force. The cavities, holes, or vessels can be connected to an inlet line to allow for the introduction of liquid into the centrifuge, and upon subjection of the spin force, can separate the liquid into different components. For example, the inlet line may be fed along or through a spindle of the centrifuge to allow liquid to enter the centrifuge. The separated portions can be removed from the centrifuge using one or more outlet lines, allowing for the separated portions to be differentially processed downstream. In some embodiments, the centrifuge can be a disk stack centrifuge and can comprise disk stack separators. The disk stack centrifuge can comprise a bowl with a plurality of conical disks. The conical disks can be closely spaced to one another to form a disk stack. The centrifuge can rotate a bowl causing the disk stack to rotate and impart a spin force to the fluid. The disk stack can allow the fluid in the bowl to be separated into a thin layer, thereby imparting the spin force throughout the fluid and reducing the settling distance of individual objects and increasing the efficiency of the separation. Rotation of the disk stack in the centrifuge can allow for a separation of the fluid into a centrate portion (or supernatant) comprising lighter (lower density) liquids and a concentrate comprising heavier (higher density) objects such as cells. The separated portions can be removed from the centrifuge using one or more outlet lines, allowing for the separated portions to be differentially processed downstream. An advantage of a disk stack centrifuge is that it can be continuously operated. An inlet can be provided on the centrifuge that continuously adds new contents into the centrifuge. Two outlet ports (one port for the supernatant or centrate, and another port for the cell comprising portion) can be used to continuously remove the separated portions from the centrifuge.

The cell density of the cell comprising portion (e.g., concentrate) can have a higher cell density than content in the bioreactor. The cell density of the cell comprising portion can be at least 1.1 fold more dense than the cell density of the bioreactor content. The cell density of the cell comprising portion can be at least 1.2 fold more dense, 1.3 fold more dense, 1.4 fold more dense, 1.5 fold more dense, 1.6 fold more dense, 1.7 fold more dense, 1.8 fold more dense, 1.9 fold more dense, 2 fold more dense, 3 fold more dense, 4 fold more dense, 5 fold more dense, 6 fold more dense, 7 fold more dense, 8 fold more dense, 9 fold more dense, 10 fold more dense, 15 fold more dense, 20 fold more dense, 30 fold more dense, 40 fold more dense, 50 fold more dense, 60 fold more dense, or more than the cell density of the bioreactor content. The cell comprising portion can have a cell concentration of at least 20 million cells per milliliter, 30 million cells per milliliter, 40 million cells per milliliter, 50 million cells per milliliter, 60 million cells per milliliter, 70 million cells per milliliter, 80 million cells per milliliter, 90 million cells per milliliter, 100 million cells per milliliter, 110 million cells per milliliter, 120 million cells per milliliter, 130 million cells per milliliter, 140 million cells per milliliter, 150 million cells per milliliter, 160 million cells per milliliter, 170 million cells per milliliter, 180 million cells per milliliter, 190 million cells per milliliter, 200 million cells per milliliter, 210 million cells per milliliter, 220 million cells per milliliter, 230 million cells per milliliter, 240 million cells per milliliter, 250 million cells per milliliter, 260 million cells per milliliter, 270 million cells per milliliter, 280 million cells per milliliter, 290 million cells per milliliter, 300 million cells per milliliter, 310 million cells per milliliter, 320 million cells per milliliter, 330 million cells per milliliter, 340 million cells per milliliter, 350 million cells per milliliter, 360 million cells per milliliter, 270 million cells per milliliter, 380 million cells per milliliter, 390 million cells per milliliter, 400 million cells per milliliter or more.

In various embodiments, content from a bioreactor is provided to a centrifuge, and a portion of the separated content is subsequently diverted back into the bioreactor. Content in the bioreactor can include cells, and a portion of the separated content that is diverted back can be a concentrate of cells that includes a different concentration of cells (e.g., about 50-100% of a cell count of the content received by the centrifuge). The portion of the content diverted back to the centrifuge can be directed via a fluidic pathway that can include a bleeding port. A bleeding port can include an outlet that “bleeds” or removes a portion of the content to another fluidic pathway. Generally, bleeding the system allows for the removal of cells or spent medium from the bioreactor. By bleeding the system, the overall cell density (amount) of the bioreactor can be reduced. Cells in a bioreactor can compete with one another for nutrients and at certain levels of cell density, the cells may adversely affect the growth of one another. Bleeding can maintain the cell density while improving the overall viability. Additionally, removal of spent media along with cells can provide for the volume of the bioreactor content to remain constant while allowing for input of fresh media. Traditional configurations of bleeding ports are typically directly connected to the bioreactor vessel. Since product of cells is secreted into the media, bleeding directly from the bioreactor vessel can result in substantial product loss. The configuration in which a centrifuge is integrated into the system and the bleed port is placed after centrifugation of bioreactor contents, as described herein, and for example with reference to FIG. 2, can reduce product loss thereby improving product yield. For example, the bleed port in system 200 can remove media from a portion that includes substantially less product. The centrifuge can separate contents into a liquid portion comprising the product and another portion comprising cells. The cell comprising portion can include substantially less product and at least a part of the cell comprising portion can be bled from the system. The bleeding port can configured to remove a predefined volume of the cell comprising portion (e.g., 238) from the fluidic pathway (e.g., 236) before the remaining volume of the cell comprising portion is transferred back to the bioreactor.

A volume of the cell comprising portion that is bled from the bioreactor system can be determined based on a volume of the bioreactor content. The bled volume can be a percentage of the volume of the bioreactor content. For example, a portion of the volume of the cell comprising portion can be removed at a rate ranging from 1% to 30% of the volume of the bioreactor content per day. A portion of the volume of the cell comprising portion can be removed at a rate of at least 1% of the volume of the bioreactor content per day. A portion of the volume of the cell comprising portion can be removed at a rate of at least 5% of the volume of the bioreactor content per day. A portion of the volume of the cell comprising portion can be removed at a rate of at least 10%, 15%, 20%, 25%, 30%, 35%, 40%, 45%, 50%, or more of the volume of the bioreactor content per day. A portion of the volume of the cell comprising portion can be removed at a rate of no more than 10%, 15%, 20%, 25%, 30%, 35%, 40%, 45%, 50%, or less of the volume of the bioreactor content per day.

Bleeding from the cell comprising portion after centrifugation can be advantageous in that product loss during bleeding is generally reduced and a bleed rate can be modulated without concern for significant product loss. Bleeding after centrifugation can be used to more efficiently control and/or modulate cell density without negatively affecting product loss. As such, the operating cell density of a system can be optimized independent of product loss.

Content that are diverted back to the bioreactor, after centrifugation, can include a higher concentration of cells compared to the concentration of cells in the bioreactor. Since the centrifugation process allows for the separation of the cell suspension into a supernatant fraction and a cell fraction, the cell fraction may include less liquid than the supernatant fraction. The cell concentration of the cell fraction can be based at least in part on the cell concentration of the content in the bioreactor and a spin force that the centrifuge exerts on content received by the centrifuge. For example, by exerting a higher spin force on a suspension of cells, more cells can be pulled away from the liquid, thereby increasing the overall number of cells in the cell fraction. Conversely, a lower spin force can allow more cells to remain in suspension, which may result in the cell fraction having a lower number of cells. The contents diverted back to the bioreactor can comprises at least 50% of a number of cells that are initially received by the centrifuge. The contents diverted back to the bioreactor can comprises at least 60%, 65%, 70%, 75%, 80%, 85%, 90%, 95%, or more, of a number of cells that are initially received by the centrifuge.

The centrifuge can include a variety of different materials. For example, in some embodiments, the centrifuge can include plastic. The centrifuge can also include metal. The centrifuge can also include ceramic. In some implementations, the centrifuge can be devoid of any metal. The centrifuge can be wholly comprised of plastic. In some examples, the centrifuge rotor or bowl can include metal or plastic or ceramic. The disk stacks of the centrifuge can include metal or plastic.

In some applications, bioreactor contamination poses a risk for the viability of bioreactor content and output. Contaminants include chemical or biological molecules that can adversely affect the purity of the resulting products. For example, a contaminant can comprise cells that do not produce a therapeutic, or generate a different therapeutic for the currently produced product. As such, the components of the systems described herein can be configured to or be subjected to processes to reduce the risk of contamination. For example, the centrifuge can be configured for single use, which may reduce the risk of potential contamination. A single use centrifuge can have several advantages over a multi-use centrifuge. For example, a multi-use centrifuge can be used for a first run of the bioreactor for generating a first therapeutic molecule, and then used for a second run for generating a second therapeutic molecule. However, the multi-use centrifuge may retain molecules from the first run and contaminate the second run with those molecules. The implementation of a single use centrifuge can reduce the risk of contamination. The centrifuge can be disposable. The centrifuge can be replaceable. For example, after the centrifuge is used to process a run of the bioreactor, the centrifuge can be disposed, and replaced by a new centrifuge for the next run of the bioreactor.

A centrifuge can provide a substantially aseptic environment. The aseptic environment can eliminate potential biological contaminants. Since cells that are processed by the centrifuge can be diverted back to the bioreactor, potential biological contaminants in the centrifuge can otherwise be diverted back to the bioreactor and contaminate the bioreactor vessel. The aseptic environment of the centrifuge can prevent contamination of the bioreactor vessel. The centrifuge can be subjected to sterilization prior to use with the bioreactor. For example, the centrifuge can be pre-sterilized via an autoclave, irradiation, or any other sterilization methods. The centrifuge can be packaged in aseptic packaging. The centrifuge can be removed from the aseptic packing and subsequently connected or coupled to the bioreactor in an aseptic environment.

A centrifuge (e.g., centrifuge 235) can be releasably coupled to other components in the system(s) described herein. For example, a bioreactor can be releasably coupled to the centrifuge. The centrifuge can be releasably coupled to a filtration unit. The releasable coupling can allow for the centrifuge to be easily replaced. For example, as described herein, the centrifuge can be a single-use centrifuge and may need to be replaced after a one-time use. The releasable coupling can facilitate the replacement of the centrifuge without substantially affecting the rest of the system. For example, the centrifuge can include an aseptic connection/disconnection device that provides an interface with other modules.

The centrifuge (e.g., centrifuge 235) can be configured to be operated continuously for a certain duration. For example, the centrifuge can be configured to be operated continuously for a duration of at least 10 days. The centrifuge can be configured to be operated continuously for a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more days. Since bioreactors can be operated continuously for a certain duration of time, the centrifuge can be operated for the same duration. The centrifuge, which can provide improved bioreactor efficiency as described herein, can be operated for the entire duration of operation of the bioreactor, which can maximize the efficiency of the bioreactor system. In some implementations, the centrifuge can operate for less of a duration of the operation of a bioreactor. For example, a centrifuge can operate for half of the overall duration of the operation of the bioreactor, and can be replaced during the operation of the bioreactor with a new centrifuge that operates for the second half of the bioreactor operation.

In various aspects, the systems described herein includes a filtration unit (e.g., filtration unit 240). The filtration unit can be used to remove impurities or allow for concentration or isolation of molecules. For example, the filtration unit can remove cells from the bioreactor content and allow for smaller macromolecules to pass through the filter and be collected. Thereby, liquid that passes through the filter can be enriched with product, while removing unwanted objects. The filtration unit can be configured to enable tangential flow filtration (TFF) to generate a perfusate and a retentate, for example. Tangential flow filtration can be performed by a fluid flow along a first axis with a filtration unit that is parallel, or at a non-orthogonal angle, to the flow axis. The fluid may flow through the filtration unit via flow forces that are non-parallel to the filtration unit, thereby filtering the fluid. Advantages of TFF can include minimal or reduced clogging or fouling of the filter, since the predominant flow axis is parallel to the filter and can remove any clogging or fouling material from the filter. The filtration system can be configured to enable alternating tangential flow filtration (ATF) to generate a perfusate and retentate. ATF filtration can be performed by a fluid flow into a filtration unit with a filter that is parallel, or at a non-orthogonal angle, to the flow axis, thereby filtering the fluid. The direction of flow can initially be in a first direction to draw fluid into the filtration unit. The fluid flow can then be reversed and allow the retentate to flow back out though the inlet channel and allowing the perfusate to flow through the filter and out the filtration unit. This reversal of fluid flow can be performed iteratively to introduce new fluid into the filtration unit and can be performed in a continual manner.

The filtration unit can be configured to filter a fluidic portion from the centrifuge. The centrifuge can generate a supernatant portion, and the supernatant portion can be filtered using the filtration unit. Bioreactor systems that include a centrifuge (as described herein) can improve filtration function by reducing the number of potentially fouling and/or clogging particles from the filtration unit. Conversely, in systems without a centrifuge, contents of the bioreactor may include a high concentration of cells, which can adversely interact with the filtration unit. With a centrifuge, bioreactor content can be separated into a cell comprising portion and a supernatant portion, and the supernatant portion, which has a lower cell density than the pre-centrifuged content, can then be provided to the filtration unit.

After providing fluid to the filtration unit, a perfusate (e.g., 252) and retentate (e.g., 262) can be generated. The perfusate is a portion that can pass through the filter, whereas the retentate is a portion that does not pass through the filter. The perfusate can be removed and collected. The perfusate can include product that is derived from the cells of the bioreactor. The retentate can be returned to the bioreactor. A third fluidic pathway (e.g., 260) can be present in the system that is configured to transfer the retentate (e.g., 262) from the filtration unit (e.g., 240) back to the bioreactor (e.g., 201) for re-use in generation of a product. Since the retentate comprises cells, the return of cells back to the bioreactor can allow for increased cell density and allow for product generation.

The perfusate can be removed at a predefined volumetric rate per day. The predefined volumetric rate can be based on a volume of the bioreactor content. For example, the predefined volumetric rate of removal of the perfusate can range from 50% to 1000% of the volume of the bioreactor content per day. The predefined volumetric rate of removal of the perfusate can be at least 50% to of the volume of the bioreactor content per day. The predefined volumetric rate of removal of the perfusate can be at least 50%, 60%, 70%, 80%, 90%, 100%, 110%, 120%, 130%, 140%, 150%, 160%, 170%, 180%, 190%, 200%, 300%, 400%, 500%, 600%, 700%, 800%, 900%, 1,000%, or more, of the volume of the bioreactor content per day.

As described herein, the overall working volume (V₃) of the bioreactor system can be held constant to allow for continuous operation. The removal of the perfusate can reduce the net working volume of the bioreactor. To maintain the working volume of the bioreactor, new media can be added at a proportional rate to counteract the loss of volume associated with the removal of the perfusate. Since there are additional outlets that can remove volume from the system (e.g., bleeding outlets), the rate of removal of perfusate can be less than the rate of new media added to the bioreactor.

Any of the systems described herein can be a closed system and can be operated in an substantially aseptic environment. The minimization of contamination throughout the process can allow for higher yield of cells, that are viable and effective for cell therapy. The system(s) described herein can include any container or consumables for the storage and manipulation of cells. The consumables can be aseptic consumables. The consumables can be single use. Single use aseptic consumables can allow for a aseptic manufacturing environment and decrease contamination of the cells at any stage in the process.

Any of the systems described herein can be fully automated systems or partially automated systems. The systems can require minimal to no user input during the processes. The fully automated systems can allow a user to initiate the system and without any addition further user input, the system can provide or generate a product. This can remove human error from the manufacturing process, and allow for savings in costs and time. Any individual process may be automated, for example, the initiation of the centrifuge, the input of fresh media, the bleeding of media, the extraction of product, etc. Processes may also be initiated manually (e.g., by a user) and can be performed in conjunction with automated processes.

The systems (or any components thereof) described herein can be configured to be operated continuously. For example, a system can be operated continuously for at least 10 days. A system can be operated continuously for at least 20 days. A system can be operated continuously for at least 30 days. A system can be operated continuously for at least 40 days. A system can be operated continuously for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, days.

Any of the systems described herein including a centrifuge may reduce product loss compared to other systems that do not use a centrifuge. Product loss of a system including a centrifuge as described can be less than about 16%. Product loss of a system including a centrifuge as described can be less than about 10%. Product loss of a system including a centrifuge as described can be less than about 10%. Product loss of a system including a centrifuge as described can be about 2%. Product loss of a system including a centrifuge as described can be no more than about 20%, 19%, 18%, 17%, 16%, 15%, 14%, 13%, 12%, 11%, 10%, 9%, 8%, 7%, 6%, 5%, 4%, 3%, 2%, or 1%, or less. Product loss can be measured, for example on a daily basis.

In various aspects, cells can be grown in any of the systems described herein. Cell content can include cells derived from immortalized cell lines. For example, cell content can include CHO (Chinese Hamster Ovary) cells or HEK293 (human embryonic kidney) cells. Cell content can include hybridoma cells. Cell content can include insect cells. Cell content can include mammalian cells. Cells content can include human cells. Cell content can include immune cells. For example, cell content can include lymphocyte, T-cells, NK cells, B cells, leukocytes, macrophages, dendritic cells, monocytes, mast cells neutrophils or other immune cells. Samples can include naïve T-cell or B-cells. Samples can include mature T-cell or B-cells.

In various aspects, cells that are grown in the bioreactor can express a polypeptide. Cells can be activated or induced to express a polypeptide. A polypeptide can be a soluble polypeptide. Cells can secrete a polypeptide into the surrounding media such that polypeptide can be isolated from the media without having to lyse the cells. For example, the soluble polypeptide can include a growth factor, cytokine, interleukin, or antibody.

In various aspects, any of the cells described herein can be genetically modified via the introduction of nucleic acids. Nucleic acids can be RNA or DNA. Cells can be transduced, transfected, or otherwise provided with nucleic acids for uptake into the cell (e.g. integration into the genome or transiently present in the cell). For example, a gene encoding an antibody can be encoded in a viral vector. A viral vector can be allowed to insert genetic material into a cell and incorporate nucleic acids from the viral vector into the cell genome. Cells can be transfected using mRNA transfection. For example, cells can be subjected to a transfection agent and an mRNA to allow uptake of the mRNA into cells. Introduction of nucleic acids into the cells can allow for the expression of exogenous polypeptides. For example, nucleic acids can include an engineered vector. A nucleic acid can encode for an antibody and can allow for the expression and secretion of the antibody.

In various aspects, a polypeptide secreted from cells can be collected as a product. The product can be an antibody or fragment thereof. For example, a product can be a monoclonal antibody, an Fc fusion protein, a nanobody, a scFv, or a Fab fragment. A product can be a binding protein, a bispecific binding protein, or an enzyme. Product collection can be performed by collecting the perfusate. Collection can include moving the perfusate to a new container. A perfusate can be subjected to additional processes of separation or isolation to generate a pure or substantially pure solution of the polypeptide. For example, the perfusate may be subjected to chromatography, such as ion exchange, size exclusion, reverse phase, or affinity chromatography. Various reagents can be introduced to the solution of polypeptides to generate formulations. The product formulations can allow the product to have improved stability, improved viability, increased shelf life, improved therapeutic efficacy, or other improved parameters.

The systems and methods described herein can be flexible in accommodating a diversity of different cells, and can generate a variety of different products based at least on the type of cells and/or the products secreted by the cells. The present disclosure is not limited to a type of cell or product and can be adapted to culture different cells by altering the media, nutrients, incubation parameters, and/or flow parameters to modulate for optimal or preferred cell densities. Similarly, the parameters of filtration, such a flow rates, pore size, or filter material, can be modified to accommodate different cell types or product types. The parameters of centrifugation can also be modified to accommodate different cell type, for example, via modification of spin speed.

### Computer Control Systems

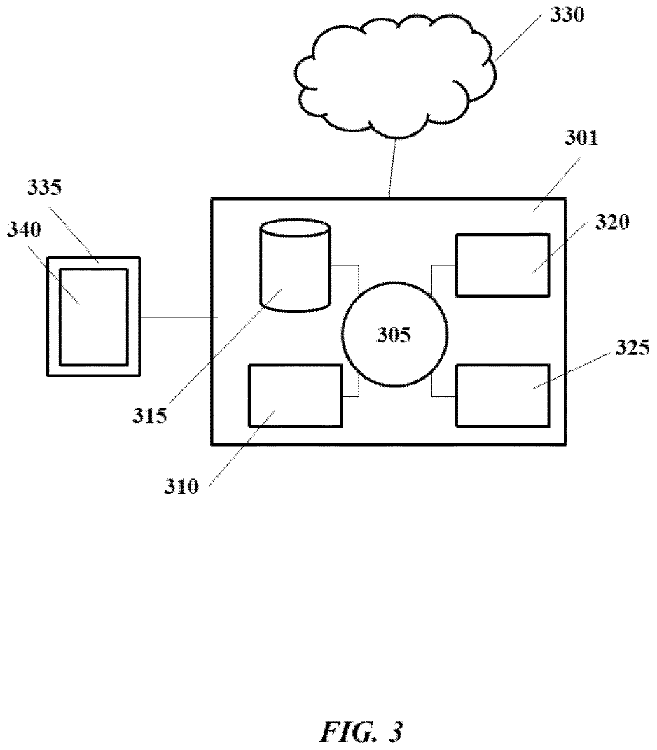

The present disclosure provides computer control systems that are programmed to implement methods of the disclosure. FIG. 3 shows a computer system 301 that is programmed or otherwise configured to perform processes described throughout this disclosure. The computer system 301 can regulate various aspects of systems or modules of the present disclosure, such as, for example, regulating the bioreactor, monitor the bioreactor parameters, controlling the flow of the contents to the bioreactor, controlling the centrifuge, or provide reports related to one or more bioreactor processes and/or productivity. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which can enable devices coupled to the computer system 301 to behave as a client or a server.

The CPU 305 can execute a sequence of machine-readable instructions, which can be embodied in a program or software. The instructions can be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., laboratory technician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 301 can include or be in communication with an electronic display 335 that comprises a user interface (UI) 340 for providing, for example, status reports of the system, data relating to the characteristic of the cells. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, determine an optimal flow of contents to the centrifuge.

### EXAMPLES

### Example 1. Generation of a Polypeptide Product from Cells in a Bioreactor System

Bioreactor 201 includes a vessel with a volume V₁. The vessel can contain media 205 of a volume V₂. Cells that are configured to express and secrete an polypeptide product (e.g., antibody) can be added to bioreactor 201 and disperse in growth media 205. Bioreactor 201 includes various components and/or devices to regulate temperature, regulate pressure, aerate the media, and/or agitate the cells, and is initiated to allow cells to grow. Cells in the bioreactor generate and secrete a polypeptide product into the media. Input line 210 can be used to add fresh media into the bioreactor 201 to provide a supply of new nutrients to the growing cells. Outlet line 230 provides a fluidic connection between the bioreactor 201 and a centrifuge 235 and can direct the cell culture 232 from the bioreactor to the centrifuge. The centrifuge 235 applies a spin force to the received content thereby generating a cell comprising portion 238 (e.g., a concentrate, pellet) and a supernatant portion 239 (e.g., centrate). The cell comprising portion 238 can be diverted back to the bioreactor 201 via fluidic pathway 236. The supernatant portion 239 that includes the polypeptide product is transferred via fluidic pathway 237 to a filtration unit 240 (e.g., a tangential flow filtration unit). The filtration unit 240 can filter the supernatant portion 239 into a perfusate portion 252 and a retentate portion 262. The filtration unit 240 can filter out additional cells from entering the perfusate 262. If the filtration unit 240 is a tangential flow filtration unit, the flow direction orthogonal to the filter may reduce fouling or clogging of the filter. The perfusate 252 includes the polypeptide product, while being substantially free of cells. The perfusate 252 can be collected via fluidic pathway 250 and the product can be isolated from the perfusate 252. The retentate 262 can be returned back to bioreactor 201. The bioreactor system includes a bleeding port or line 220 which can be used to remove a portion 222 of the contents of the bioreactor system, for example as waste. Bleeding system content can facilitate the removal of spent media as well as removal of excess cells to maintain a suitable cell density in the bioreactor for high cell viability. The bleeding port 220 can be connected to the fluidic pathway 236 that transfers the cell comprising portion 238 back to the bioreactor 201. Since the cell comprising portion 238 includes less product than the cell culture 232 transferred from the bioreactor 201 to the centrifuge 235, the loss of media from the bleeding port can reduce product loss. Additionally, since the centrifuge 235 diverts the cell comprising portion 238 away from the filtration unit 240, the cell count of the supernatant portion 239 transferred to the filtration unit 240 is significantly lower than the cell count of the cell culture 232 transferred from the bioreactor 201 to the centrifuge 235. Since fewer cells are transferred to the filtration unit 240, less fouling and clogging of the filtration unit 240 may occurs, thereby extending the life cycle of the filtration unit 240. The rates at which media is added to the bioreactor and content removed from the bioreactor system can be balanced to maintain a constant working volume in the bioreactor system. For example, fresh media can be added at a rate of 120% V₁/day and to maintain a constant volume, 100% V/day can exit the system as perfusate and 20% V₁/day can exit the system via the bleeding port (e.g., as waste). The overall product loss may be 2% of total production for a system with 20% cell density in the bioreactor, and 90% cell density in the cell comprising portion derived from the centrifuge. Product can be isolated and formulated for suitable use for treatment of a subject, and can be provided for a subject for treatment.

While preferred embodiments have been shown and described herein, it will be obvious to those skilled in the art that such embodiments are provided by way of example only. It is not intended that the disclosure be limited by the specific examples provided within. The descriptions and illustrations of the embodiments herein are not meant to be construed in a limiting sense. Numerous variations, changes, and substitutions will now occur to those skilled in the art without departing from the disclosure. Furthermore, it shall be understood that aspects of the disclosure are not limited to the specific depictions, configurations or relative proportions set forth herein which depend upon a variety of conditions and variables. It should be understood that various alternatives to the embodiments described herein can be employed in practice. It is therefore contemplated that the disclosure shall also cover any such alternatives, modifications, variations or equivalents. It is intended that the following claims define the scope of the disclosure and that methods and systems within the scope of these claims and their equivalents be covered thereby.

1. A system comprising:
a centrifuge in fluidic communication with a bioreactor, wherein the centrifuge is configured to receive and separate content from the bioreactor into a first portion and a second portion, wherein a composition of the first portion is different from a composition of the second portion;
a first fluidic pathway configured to transfer the first portion from the centrifuge back to the bioreactor; and
a second fluidic pathway configured to transfer the second portion from the centrifuge to a filtration unit.

2. The system of claim 1, wherein the centrifuge comprises:
an inlet port configured to receive content from the bioreactor;
a first outlet port connected to the first fluidic pathway; and
a second outlet port connected to the second fluidic pathway.

3-6. (canceled)

7. The system of claim 1, wherein the second portion comprises a supernatant.

8-10. (canceled)

11. The system of claim 1, wherein the centrifuge comprises disk stack separators and is configured to be operated continuously for an extended time duration of at least 10 days.

12. (canceled)

13. The system of claim 1, wherein the centrifuge is releasably coupled to the bioreactor.

14. The system of claim 1, wherein the centrifuge is releasably coupled to the filtration unit.

15. (canceled)

16. The system of claim 1, wherein the first fluidic pathway comprises a bleeding port, and wherein the bleeding port is arranged to remove a predefined volume of the first portion from the first fluidic pathway before a remaining volume of the first portion is transferred back to the bioreactor.

17. (canceled)

18. The system of claim 16, wherein the predefined volume of the first portion is determined based on a volume of the content in the bioreactor.

19-20. (canceled)

21. The system of claim 1, further comprising the bioreactor, wherein the bioreactor is configured to receive media at a predefined rate per day, and wherein the predefined rate is based on one or more of a volume of the content in the bioreactor, a predefined volume of the first portion that is removed through bleeding, or a volume of perfusate generated by the filtration unit.

22-25. (canceled)

26. The system of claim 1, further comprising the filtration unit, wherein the filtration unit is configured to enable tangential flow filtration (TFF) of the second portion to generate a perfusate and a retentate.

27. (canceled)

28. The system of claim 26, wherein the perfusate is removed at a predefined volumetric rate per day, and wherein the predefined volumetric rate is based on a volume of the content in the bioreactor.

29-30. (canceled)

31. The system of claim 26, further comprising a third fluidic pathway configured to transfer the retentate from the filtration unit back to the bioreactor for re-use in production of a product.

32. A method comprising:
performing centrifugation on content received from a bioreactor, thereby separating the received content into a first portion and a second portion, wherein a composition of the first portion is different from a composition of the second portion; and
transferring the first portion back to the bioreactor, and transferring the second portion to a filtration unit.

33. (canceled)

34. The method of claim 32, wherein a cell concentration of the first portion is higher than a cell concentration of the content in the bioreactor, and wherein the cell concentration of the first portion is based at least on the cell concentration of the content in the bioreactor and a spin force exerted by the centrifugation on the received content.

35. (canceled)

36. The method of claim 32, wherein the second portion comprises supernatant.

37. (canceled)

38. The method of claim 32, further comprising removing a predefined volume of the first portion before transferring a remaining volume of the first portion back to the bioreactor, wherein the predefined volume of the first portion is determined based on a volume of the content in the bioreactor.

39-40. (canceled)

41. The method of claim 32, wherein the bioreactor receives media at a predefined rate per day, and wherein the predefined rate is based on one or more of a volume of the content in the bioreactor, a predefined volume of the first portion that is removed through bleeding, or a volume of perfusate generated by the filtration unit.

42-44. (canceled)

45. The method of claim 32, further comprising: performing tangential flow filtration (TFF) on the second portion using the filtration unit to thereby generate a perfusate and a retentate.

46. The method of claim 45, further comprising removing the perfusate at a predefined volumetric rate, wherein the predefined volumetric rate is based on a volume of the content in the bioreactor.

47-48. (canceled)

49. The method of claim 45, further comprising transferring the retentate from the filtration unit back to the bioreactor for re-use in production of a product.

50-54. (canceled)
